(19) United States
(12) Patent Application Publication
(10) Pub. No.: US 2020/0178820 A1
(43) Pub. Date: Jun. 11, 2020

(54) METHOD AND ARRANGEMENT FOR CONTINUOUSLY ESTIMATING BLOOD PRESSURE

(71) Applicant: Bittium Biosignals Oy, Kuopio (FI)

(72) Inventor: Antti RUHA, Oulu (FI)

(21) Appl. No.: 16/216,251

(22) Filed: Dec. 11, 2018

(51) Int. Cl. A61B 5/024; A61B 5/0245; A61B 5/021; A61B 5/0285

(52) U.S. Cl. CPC A61B 5/02416; A61B 5/0245; A61B 5/02255; A61B 5/0285; A61B 5/02108

(57) ABSTRACT

Disclosed is a method and an arrangement implementing the method for continuously estimating blood pressure. The method includes receiving an electrocardiography signal and a first photoplethysmography signal measured from the person, extracting values of a timing parameter set based on timings of the electrocardiography signal and the photoplethysmography signal, and calculating at least two intermediate estimates of the blood pressure of the person with linear regression models that model are based on different timing parameter subsets of the timing parameter set. At least one final estimate is then calculated on the basis of the intermediate estimates.

### FIELD

The invention relates to forming an estimated of blood pressure, and in particular, to methods and arrangements for non-invasively and continuously estimating the blood pressure.

### BACKGROUND

Blood pressure is conventionally been measured with a blood pressure meter (i.e. sphygmomanometer) comprising an inflatable cuff and a pressure sensor indicating the pressure of the cuff. The cuff is used to collapse and then release an artery under the cuff in a controlled manner. The cuff and the pressure sensor are used in conjunction with means configured to determine at what pressure value blood flow is just starting and at what pressure value it is unimpeded.

However, a conventional blood pressure meter may be uncomfortable to wear and use over longer periods and difficult to use in everyday situations. The measurement process may be disruptive due to the noise and discomfort it causes. This may make the use of a blood pressure cuff impractical during sleep, for example. Due to the operating principal and its disruptive nature, a blood pressure cuff can be measured only from time to time in practice. While a small servo-controlled cuff around a finger can enable a practically continuous measurement, it is still uncomfortable to wear and use over longer periods and may be difficult to use in everyday situations.

Blood pressure may also be estimated by determining transit time of an arterial pulse wave between two points in the artery. The velocity of the arterial pulse wave increases in response to an increase in the blood pressure. By detecting the transit time, an estimate of the velocity of the arterial pulse wave and therefore also the blood pressure can be formed. However, a non-invasive measurement of the progress of an arterial pulse in an artery may be difficult because the measurement signals may have a low signal-to-noise ratio. Further, characteristics of the measured signals may vary significantly from person to person. Therefore, it may be very difficult to reliably form an estimate of the blood pressure of a person.

### SUMMARY

An object of the present disclosure is to provide a non-invasive method for continuously estimating blood pressure and a system for implementing the method so as to alleviate the above disadvantages. The object of the disclosure is achieved by a monitoring method and an estimation method which are characterized by what is stated in the independent claims. The preferred embodiments of the disclosure are disclosed in the dependent claims.

In a monitoring method according to the present disclosure, an electrocardiographic (ECG) signal and at least one photoplethysmographic (PPG) signal are measured from a person. The monitoring method then utilizes an estimation method according to the present disclosure in order to form an estimate of blood pressure based on the measured signals.

In the estimation method, values of a plurality of timing parameters related to the arterial pulse wave may be extracted from the measured ECG and PPG signals. The timing parameters may represent different aspects of the arterial pulse wave. For example, a pulse transition time (PTT) of the arterial pulse wave may be calculated between the ECG signal and the PPG signal. Further, a rise time, a fall time and other timing points may be determined from the waveform of the PPG signal. An estimation method according to the present disclosure may further comprise measuring a second PPG signal. This enables a variety of PTTs to be calculated: ECG to first PPG, ECG to second PPG, and first PPG to second PPG.

An estimation method according to the present disclosure may also comprise decomposing at least one PPG signal into wave components. Timing values may then be calculated for a pulse transition time between a forward wave component and a reflection wave component of the PPG signal, and/or for a pulse transition time between a first reflection wave components and a second reflection wave component of the PPG signal.

The extracted values of the timing parameters are fed to a multiple linear regression model that is configured to estimate the blood pressure of the person. Thus, the estimated blood pressure is based on more than one timing parameter. This improves the reliability of the estimation and provides means for adjusting the estimation to different measurement characteristics. Because timing characteristics of the arterial pulse wave may vary significantly between different populations or demographics (differentiated e.g. by age, weight, or gender), or even between persons with a same population, it may be advantageous to be able to adjust the regression model accordingly. Therefore, the estimation method preferably comprises at least one calibration step for determining the calibration timing values for the timing parameters. These calibration timing values may be coupled with calibration blood pressure measurements which may be performed in the conventional way, e.g. with a blood pressure cuff. In this manner, the regression model can be personalized, even for each person individually if necessary.

Another aspect of the estimation method according to the present disclosure is improvement of quality of the PPG signal. The quality of the PPG pulse may be improved by coherent averaging with QRS complex as trigger signal, for example. This allows PPG measurements from body locations where beat-to-beat signal quality is otherwise too low for a successful estimation. The PPG may be measured from alternative locations, such as a fingertip or an ear lobe. However, a particularly preferable location is the chest. Measurements from the chest provide access to the closest estimate of central blood pressure that typically is of most interest in monitoring phenomena related to the heart and to big arteries. Measurements from the chest may also be advantageous as they enables compact implementation of the device in a single housing.

The above-described monitoring method and estimation method may be implemented in various ways. For example, the methods may be implemented with the aid of a measurement device comprising sensors configured to measure an ECG signal and a PPG signal from the person. The measurement device may be a stand-alone device, i.e. it may further comprise a processing unit configured to implement the estimation method according to the present disclosure. Alternatively, the measurement device may be configured to send the measured signal data to a remote estimation system. The remote estimation system may be implemented (at least partially) as a cloud computing system, for example.

The method and system according to the present disclosure provide a new, reliable way for continuously and non-invasively monitoring the blood pressure of a person. The estimation method may be based on non-invasive measurement of electric signals, thereby avoiding the uncomfortable use of air pump, related piping and the cuff. This allows a small form factor and power consumption and is suitable for a portable/wearable device. The estimation method enables measurement from other body locations than fingertip and ear lobe, such as the chest.

### BRIEF DESCRIPTION OF THE DRAWINGS

In the following the invention will be described in greater detail by means of preferred embodiments with reference to the attached drawings, in which

FIG. 1 shows a simplified flow diagram of a monitoring method according to the present disclosure;

FIG. 2 shows a simplified flow diagram of an embodiment of the estimation method according to the present disclosure;

FIG. 3 shows a simplified flow diagram of an exemplary embodiment of extracting the values of timing parameter set;

FIG. 4 shows a simplified block diagram of exemplary embodiment of a measurement arrangement for continuously estimating blood pressure; and

FIG. 5 shows a simplified block diagram of another exemplary embodiment of a measurement arrangement for continuously estimating blood pressure.

### DETAILED DISCLOSURE

The present disclosure describes a monitoring method for continuously and non-invasively monitoring blood pressure and an arrangement implementing the monitoring method. The monitoring method according to the present disclosure is based on a combined use of electrocardiography (ECG) and photoplethysmography (PPG) signals. The method enables long-term measurement and estimation of blood pressure and, more generally, of cardiovascular condition. In particular, the method enables detection of changes in the blood pressure during a monitoring period.

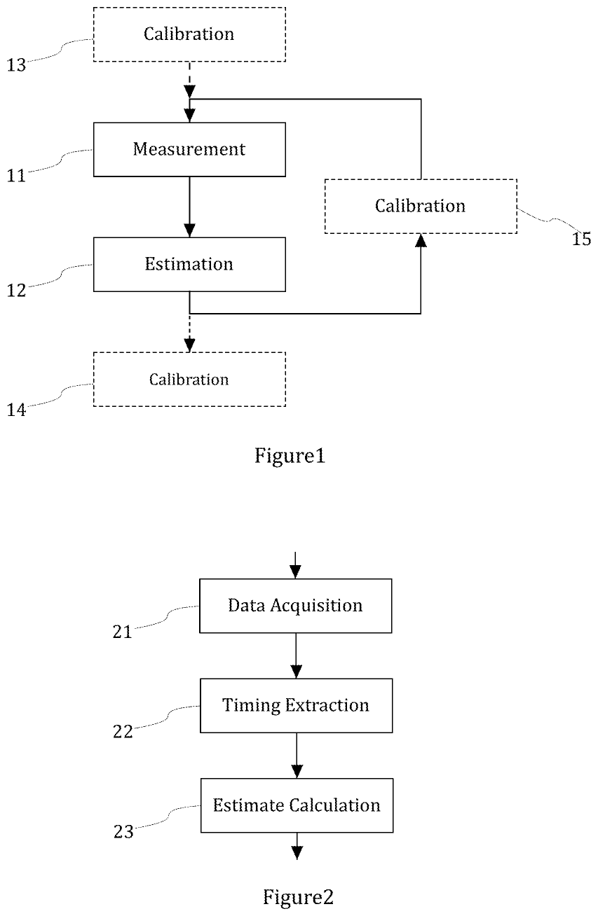

In the monitoring method according to the present disclosure, an ECG signal and at least a first PPG signal are measured from a person. FIG. 1 shows a simplified flow diagram of a monitoring method according to the present disclosure. In FIG. 1, the ECG and PPG signals are measured in a measurement phase 11. In an estimation phase 12, an estimate of the blood pressure is formed on the basis of the ECG and PPG signals by using an estimation method according to the present disclosure. The monitoring method may also include calibration which is shown as optional calibration phases 13, 14, and 15 (drawn with dashed lines) that may occur before or after the estimation method, or between estimation updates.

In the context of the present disclosure, “estimating blood pressure” may be understood as forming an estimate of an absolute value of the blood pressure or as forming an estimate of a change in the blood pressure. Further, in the context of the present disclosure, “continuously estimating blood pressure” is intended to be understood as performing the estimation process in real time such that an estimate of the blood pressure can be updated at least once per minute, or even at each heart beat (e.g. at least once per second), if necessary.

In order to achieve a continuous estimation of blood pressure, the present disclosure describes an estimation method that comprises receiving an ECG signal measured from a person, receiving a first PPG signal measured from the person, and extracting values of a timing parameter set based on timings of the ECG signal and the PPG signal. In this context, a timing parameter set is a pre-defined group of timing parameters that are being used in the estimation method. In this context, a timing parameter represents a specific feature of the arterial pulse wave for which a timing value can be determined. For example, the ECG and PPG signals may be used to derive a PTT and other timing and waveform features of PPG. The PTT represent a transit time of an arterial pulse wave between two points in the artery. Since the velocity of the arterial pulse wave increases in response to an increase in the blood pressure, the transit time can be used as an indicator of the blood pressure. However, positioning of the PPG measurement may have a significant effect on the signal quality. For example, while it can be relatively easy to detect a PPG signal from a wrist or a fingertip and determine a PTT based on said PPG signal, determining a PTT from an ECG signal and a PPG signal measured from the chest may be significantly more difficult. Because the propagation distance from heart to PPG pickup point at the chest is short, a small PTT value is detected. Further, because changes measured from surface of the chest, the signal may have a too low signal-to-noise ratio. Therefore, it may be preferable to form the estimate of the blood pressure based at least partially on values of timing parameters representing PPG wave slope rise time (and/or fall time) when the PPG is measured from the chest.

The estimation method may also implement waveform decomposition. An arterial pulse wave reflects at joints of large arteries, such as renal and femoral arteries. Therefore, a PPG signal may be measured from the chest, for example, and changes in the PPG waveform may be determined based on a PPG waveform decomposition into forward (percussion) and reflected wave components. Relative timings of the wave components may then me used as indicators for estimating the blood pressure.

In some embodiments, the estimation method may utilize a second PPG signal. For example, the estimation method may receive to have an ECG signal and two PPG signals measured from two different points, such as chest and abdomen. In this manner, a PTT may be measured between two PPG waveforms instead of, or in addition to, a PTT between an ECG and a PPG. This may be preferable, since the two PPG waveforms represent real pulse wave propagation over a distance. Further, it can be assumed that peripheral circulation affects both PPGs the same way, and therefore, effects to the values of the timing parameters between the two PPGs caused by the peripheral circulation are effectively cancelled out.

While it may be possible to formulate an estimate of the blood pressure based on one timing parameter alone, the reliability of the estimation can be significantly improved by combined use of a plurality of timing parameters. This also provides means for adjusting the estimation to different measurement characteristics. Thus, in the estimation method according to the present disclosure, the timing parameter set comprises at least two timing parameters, and the method comprises calculating at least two intermediate estimates of the blood pressure of the person with linear regression models based on different timing parameter subsets of the timing parameter set. At least one final estimate of the blood pressure may then be calculated on the basis of the intermediate estimates. The at least one final estimate may include an estimate of systolic blood pressure and/or an estimate of diastolic blood pressure, for example.

FIG. 2 shows a simplified flow diagram of an embodiment of the estimation method according to the present disclosure. The estimation method in FIG. 2 comprises three phases: a data acquisition phase 21, where the ECG and PPG signals are received by the estimation method; a timing extraction phase 22, where the values of the timing parameters are extracted; and an estimate calculation phase 23, where the intermediate estimates and the final estimate is calculated. In the following, the above-mentioned phases of the estimation method according to the present disclosure are discussed in more detail.

In an estimation method according to the present disclosure, the timing extraction phase may comprise pre-processing the ECG and PPG signals, detecting a QRS complex in the ECG signal, extracting ECG and PPG waveform cycles from the ECG and PPG signals, producing averaged ECG and PPG waveforms based on the extracted ECG and PPG waveform cycles, and determining the values of the timing parameter set on the basis of the averaged ECG and PPG waveforms.

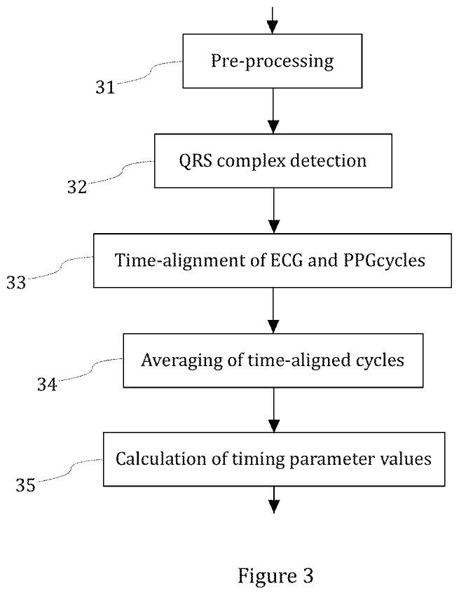

FIG. 3 shows a simplified flow diagram of an exemplary embodiment of extracting the values of timing parameter set. In FIG. 3, the ECG and PPG signal are pre-processed in step 31. The ECG and PPG signals may be pre-processed by filtering them with a digital bandpass filter, for example. It may be preferable to form the filter to have linear phase. The filter may be a FIR or forward-backward IIR filter, for example.

The QRS complex is detected in step 32 in FIG. 3. Detection of the QRS complex may be based on known detection techniques, e.g. Pan-Tompkins method. However, it may be desirable to further improve known techniques. For example, while using the R peak as a QRS fiducial point may be sufficient in some applications, a single extracted R peak point may be prone to timing jitter error as a fiducial point, especially if the sampling frequency is low (for example 350 Hz gives 4ms timing jitter). Therefore, it may be preferable to calculate a “centre of gravity” of the parts of the QRS complex above a threshold level in order to define QRS complex fiducial point. This kind of approach is less affected by timing jitter, noise and QRS waveform changes (due to breathing etc.) than a single point extraction.

In order to extract ECG and PPG waveform cycles, the ECG and PPG signals may be time-aligned with respect to the detected QRS complex. In FIG. 3, the ECG and PPG waveform cycles are time-aligned in step 33. The time-aligned cycles may be stored and similarity between cycles estimated (with a correlation coefficient or similar measure).

Quality of PPG waveform may be significantly improved by coherent averaging with the QRS complex acting as a trigger signal. This may be preferable, since it allows measurement of a PPG signals from body locations, such as the chest, where beat-to-beat signal quality may otherwise be too low for successful estimation. This can provide access to the closest estimate of central blood pressure that is of most interest in monitoring heart and big arteries related phenomena. In step 34 in FIG. 3, averages of ECG and PPG waveform cycles are formed. Known averaging techniques may be used, for example.

Weighting coefficients of the average may be adjusted on the basis of a quality (e.g. in the form of variance) of at least one of the ECG and PPG signals. The weighting coefficients may be used on the stored ECG frames and PPG frames to weight a beat signal in the averaging for achieving a robust averaging. A model of ECG waveform cycle may be created with the averaging, and this model may be updated with rate based on similarity of collected EGC cycles. This improves noise tolerance. Similarly, a model of PPG waveform cycle may be created and updated continuously with the collected PPG cycles by coherent averaging with QRS detection instant as fiducial point. This improves PPG signal quality by a significant amount and may be important as extraction from a single wave is in many cases very noisy. In other words, the waveform averaging does not have to rely on fixed weighting coefficients only.

Recursive waveform averaging is preferably used in the estimation method according to the present disclosure. Update coefficient may be made dependant on waveform similarity of subsequent frames. In other words, near identical waveforms leads to fast update, while differing waveforms (due to noise) lead to slow update. In this manner, the waveform averaging is able to adapt to signal quality and makes waveform averaging robust against noise.

The values of the timing parameter set are determined on the basis of the averaged ECG and PPG waveforms in step 35 in FIG. 3. The values of the timing parameter set may be based on the averaged waveforms. A selection of pre-defined reference points in a PPG waveform may be used. Minimums and maximums in the PPG waveforms are possible options to be used as reference points. However, timings of the minimums and maximums may be very sensitive to noise and waveform variations (due to breathing, reflection waves, for example). As a result, the minimums and maximums may not serve as good timing references. Therefore, it may be preferable to use inclined (non-flat) points of the waveform as timing reference points. For example, timings of a 10%, a 90%, and a 50% threshold level may be used as timing points (other threshold levels may be used as well). The 10% and 90% threshold level are much more stable for representing wave timings than the minimum and the maximum.

In the estimate calculation phase of the estimation method according to the present disclosure, a plurality of (i.e. at least two) intermediate estimates of the blood pressure may be calculated with linear regression models. A final estimate may then be calculated on the basis of the intermediate estimates.

The regression models may be based on different timing parameter subsets of the timing parameter set. Each linear regression model may have at least one explanatory variable that is weighted by at least one regression model coefficient. Said at least one explanatory variable may represent a deviation between a value of a timing parameter in a timing parameter subset and a corresponding calibration timing value determined based on a calibration sample set.

The calibration sample set represents blood pressure characteristics of the person and may be based on calibration measurement data of the person, The at least one regression model coefficient is also based on the calibration measurement data. The calibration sample set may comprise a plurality of calibration samples. Each sample may comprise a blood pressure value (or blood pressure values, such as diastolic and systolic blood pressure values) measured at different calibration points. Thus, each sample may represent a specific calibration point. The calibration blood pressure values may have been measured with a blood pressure cuff, for example. Each sample may further comprise a plurality of calibration timing parameter values coupled with the calibration blood pressure value(s) at the calibration point of the sample. Each calibration timing parameter value of a sample may represent a measured value of a particular timing parameter at the calibration point.

For example, at least one intermediate estimate may be calculated with a multiple linear regression model that is in the form of a weighted sum of a plurality of explanatory variables, wherein each explanatory variable is weighted by a regression model coefficient. The multiple linear regression model may be of following type, for example:

BP=BP_{ref}[1+k_{cor,1}·(t_{meas,1}−t_{ref,1})/t_{ref,1}+k_{cor,2}·(t_{meas,2}+t_{ref,2})/t_{ref,2}+ . . . ], (1)

where BP_{ref} is a single averaged calibration blood pressure value, e.g. in the form of an average of the plurality of calibration blood pressure values in a calibration sample set as defined above. BP_{ref} may represent diastolic blood pressure or systolic blood pressure, for example. t_{meas,1}, t_{meas,2}, . . . are the values of the timing parameters used in the regression model. t_{ref,1}, t_{ref,2}, . . . are averaged calibration timing parameter values corresponding with the timing parameters used in the regression model. Each averaged calibration timing parameter value t_{ref,1}, t_{ref,2}, . . . may represent an average of values of a particular timing parameter at different calibration blood pressure values in the calibration sample set. The averaged calibration timing parameter value t_{ref,1}, t_{ref,2}, . . . correspond with the BP_{ref} value. k_{cor,1}, k_{cor,2}, . . . are linear regression coefficients representing blood pressure dependency on timing. In the equations of the present disclosure are scalar, variables are denoted in italics. Operator “·” denotes multiplication, “+” denotes a sum, “−” denotes subtraction, and “/” denotes division.

The timing parameter subset may comprise at least two pulse transition times (PTT) between the signal ECG and the first PPG signal, for example. Each PTT uses a different threshold level of the PPG waveform as a timing reference point. The multiple regression model then uses the at least two PTTs as explanatory variables.

PTTs for reference point at different the threshold levels correlate with different aspects of blood pressure. For example, a PTT calculated for a threshold level (e.g. 10% threshold level) close to the diastolic blood pressure (i.e.

the minimum of the PPG waveform) correlates strongly with the diastolic pressure and correlates only weakly with the systolic pressure. In contrast, a PTT calculated for a threshold level (e.g. 90% threshold level) close to the systolic pressure (i.e. the maximum of the PPG waveform) correlates strongly with systolic pressure but only weakly with the diastolic pressure. Thus, the coefficients of each regression model may be adapted to reflect the systolic blood pressure, the diastolic blood pressure, or both, for example. In order to improve reliability of the estimation, multiple intermediate estimates (of the systolic blood pressure, diastolic blood pressure, or both) may be calculated with this kind of multiple linear regression models. Each model may be based on its own timing parameter subset. A final estimate may be calculated as an average, median, or weighted average of the intermediate estimates, for example. Preferably the final estimate is calculated as a median of the intermediate estimates.

Alternatively, or in addition, at least one intermediate estimate may be calculated with a timing parameter subset of at least one of the linear regression models comprises a time difference between two different PTTs between the signal ECG and the first PPG signal. The PTTs are based on different threshold levels of the PPG waveform as a timing reference points. These threshold levels may represent different points of a rising (or falling) slope of the PPG waveform, and thus the time difference may correlate with the slope rise time. The regression model may use the time difference as the sole explanatory variable, for example. In order to improve reliability of the estimation, multiple intermediate estimates may be calculated with this kind of linear regression models. Each model may be based on its own timing parameter subset. A final estimate may be calculated as an average, median, or weighted average of the intermediate estimates, for example. Preferably the final estimate is calculated as a median of the intermediate estimates. In order to enable the personalized calibration, an estimation method according to the present disclosure may comprise receiving the calibration measurement data from an external source. The calibration measurement data may comprise calibration values of the blood pressure of the person and values of calibration timing values corresponding the values of the blood pressure.

By using calibration measurements from an individual, a personalized linear estimation function can be formed. The personalized linear estimation function is not merely based on a population or demographics function. Instead, the personalized function can be considered to represent blood pressure function characteristics of an individual. However, in some embodiments of the estimation method according to the present disclosure, initial function coefficients may be set based on a population group or demographics group that the individual belongs to. For example, pulse velocity and the PTT are age-dependent (due to stiffness of arteries increasing with age). This information can be utilized when determining initial function coefficients for the individual.

In order to be able to calculate the regression model coefficients and personalize the regression model, a plurality of calibration points (e.g. in the form of calibration measurements) may be required. Some accuracy can be achieved and changes in the blood pressure can be detected even with only two points. However, the more calibration points, the more accurate model is. Therefore, three or more calibration points are preferably used. Different calibration points can be determined by taking calibration measurements from a person in different postures/orientations, such as sitting or standing, for example.

Another aspect of individual calibration of the estimation is improvements in data cleaning (and utilization) of the measurement data. If only common population-based or demographics-based sanity checks are used, the limits may not be well tuned for personal characteristics of the individual, thereby possibly compromising personal performance of the estimation. In the estimation method according to the present disclosure, it is therefore preferable to only use broad-ranged sanity checks (non-critical for performance), and rely mostly on median and Kalman-type filtering and dropping outliers to clean measurement data (like heart rate, RR interval, PTT values and calibration blood pressure measurements). For example, data below 5% and above 95% may be discarded. As a result of the personalized calibration, the blood pressure estimation is more robust to noise and measurement artefacts, and, at the same time, is fitted to a person and his/her physiological characteristics in best possible way.

As shown in FIG. 1, the calibration may occur at different instants during the monitoring method. Calibration measurements may be performed in calibration phases 13, 14 and/or 15 before, during, and/or after the estimation phase 12 in the monitoring method. Thus, the estimation method according to the present disclosure may receive the calibration measurement data before or after the extraction of the values of the timing parameter set, for example. The monitoring method may implement co-use of cuff measurements and the estimation method according to the present disclosure. PPG measurements may be continuous and a cuff device may be used to measures blood pressure periodically (at a predefined interval) to get/update a calibration result.

Alternatively, or in addition, the estimation method may comprise receiving the calibration measurement data during the third phase (i.e. the extraction of the values of the timing parameter set) or between subsequent iterations of the third phase. The PPG measurement may be used to detect and indicate a change in circulation (via changes in PTT, either in one or more PPG channels) and cause the cuff device to make a measurement Thus, the estimation method may comprise detecting a change in the values of the timing parameter set and comparing the change with a set limit. If the change exceeds the set limit, a trigger signal may be generated, causing the calibration measurements to be performed in order to update the calibration measurement data. Updated calibration measurement data resulting from the calibration measurements may be received, and the calibration sample set and thus also the regression model coefficients may be updated on the basis of the updated calibration measurement data.

By using both the conventional blood pressure measurement (e.g. with a cuff) and the estimation method according to the present disclosure, a high-accuracy, continuous monitoring of the blood pressure can be achieved without significantly compromising the comfort (and/or sleep quality) of the user.

In the following, some timing parameters for an estimation method according to the present disclosure are discussed in reference to exemplary embodiments. The exemplary embodiments all implement the data acquisition phase, the timing extraction phase and the estimate calculation phase of the estimation method according to the present disclosure as discussed above.

In a first exemplary embodiment, the timing parameter set comprises at least three timing parameters: a first PTT t₁ between the signal ECG and a reference point at a 90% threshold level of a rising slope first PPG signal, a second PTT t₂ between the signal ECG and a reference point at a 50% threshold level of the rising slope first PPG signal, and a third PTT t₃ between the signal ECG and a reference point at a 10% threshold level of the rising slope first PPG signal.

As discussed above, the threshold levels correlate differently with the systolic blood pressure (SBP) and diastolic blood pressure (DBP). In the first exemplary embodiment, t₁, t₂, and t₃ are assumed to represent composite blood pressures BP₁, BP₂, and BP₃, respectively, wherein the systolic blood pressure SBP and diastolic blood pressure DBP affect BP₁, BP₂, and BP₃ as follows:

BP₁=0.9·SBP+0.1·DBP, (2)

BP₂=0.5·SBP+0.5·DBP, (3)

BP₃=0.1·SBP+0.9·DBP, (4)

Because BP₁, BP₂, and BP₃ correlate with t₁, t₂, and t₃, the above equations (2)-(4) can be written as follows:

k₁·fₜ(t_{1,n})=0.9·SBP(n)+0.1·DBP(n), (5)

k₂·fₜ(t_{2,n})=0.5·SBP(n)+0.5·DBP(n), (6)

k₃·fₜ(t_{3,n})=0.1·SBP(n)+0.9·DBP(n), (7)

wherein

fₜ(t_{i,n})=(t_{i,n}−\hat{t}_{i,m})/\hat{t}_{i,m}, (8)

wherein i represents the index for the PTTs t₁, t₂, and t₃, n represents nth calibration sample in a set of m calibration samples, and \hat{t}_{i,m} represents the average of ith PTT calculated with the m samples. The coefficients k₁, k₂, and k₃ may be determined with known line-fitting methods, or as follows, for example:

cov(BPᵢ, fₜ(tᵢ))/var(fₜ(tᵢ)), (9)

wherein cov is covariance and var is variance, and BPᵢ is ith composite blood pressure.

With three PTTs (t₁, t₂, and t₃), three equation pairs can be formed in the first exemplary embodiment. With these equation pairs, estimates of relative changes (SBPᵣₑₗ and DBPᵣₑₗ) in the systolic blood pressure SBP and diastolic blood pressure DBP can be solved. For the PTTs (t₁, t₂, and t₃ as defined above, the equation pairs for the relative changes in can be as follows

SBPᵣₑₗ₁₃=1.125·k₁·fₜ(t₁)−0.125·k₃·fₜ(t₃), (10)

DBPᵣₑₗ₁₃=−0.125·k₁·fₜ(t₁)+1.125·k₃·fₜ(t₃), (11)

SBPᵣₑₗ₂₃=2.125·k₂·fₜ(t₂)−1.25·k₃·fₜ(t₃), (12)

DBPᵣₑₗ₂₃=−0.25·k₂·fₜ(t₂)+1.25·k₃·fₜ(t₃), (13)

SBPᵣₑₗ₁₂=1.25·k₁·fₜ(t₁)−0.25·k₂·fₜ(t₂), (14)

DBPᵣₑₗ₁₂=−1.25·k₁·fₜ(t₁)+2.125·k₂·fₜ(t₂), (15)

By using the equations (10)-(15) above, groups of intermediate estimates of the systolic and diastolic blood pressure can be formed:

SBP₁₃=SBP_{ref}·(1+SBPᵣₑₗ₁₃), (16)

SBP₂₃=SBP_{ref}·(1+SBPᵣₑₗ₂₃), (17)

SBP₁₂=SBP_{ref}·(1+SBPᵣₑₗ₁₂), (18)

DBP₁₃=DBP_{ref}(1+DBPᵣₑₗ₁₃), (19)

DBP₂₃=DBP_{ref}(1+DBPᵣₑₗ₂₃), (20)

DBP₁₂=DBP_{ref}(1+DBPᵣₑₗ₁₂), (21)

wherein SBP_{ref} and DBP_{ref} are averages of the calibration blood pressure values of systolic and diastolic blood pressure in the calibration samples as discussed in relation to Equation (1) above. Equations (16) to (18) each give an intermediate estimate of the systolic blood pressure while Equations (19) to (21) each give an intermediate estimate of the diastolic blood pressure. This redundancy can be utilized in the first exemplary embodiment. Final estimates of the systolic of the diastolic blood pressures may be calculated as averages, medians, or weighted averages of the respective intermediate estimates, for example. Preferably the final estimates are calculated as medians of the intermediate estimates.

In a second exemplary embodiment, rise (and/or fall) times may be utilized as primary measures and the first PTT as secondary measure, because the PTT may be sensitive to several disturbing factors, like body orientation and masked by heart's PEP (pre-ejection period, time between sinus excitation to mechanical contraction and blood output from heart).

The rise (and/or) fall times may be detected in the form of time differences between different, predetermined points on a slope of the waveform. For example, in the following, three rise times t₁₂, t₂₃, and t₁₃ are calculated on the basis of the PTTs t₁, t₂, and t₃ as defined in the first exemplary embodiment. Each of the three rise times t₁₂, t₂₃, and t₁₃ represents a time difference between two PTTs selected from t₁, t₂, and t₃, so that t₁₂=t₂, t₂₃=t₂−t₃, and t₁₃=t₃. The PTTs t₁, t₂, and t₃ represent 90%, 50%, 10% threshold levels, respectively, and therefore, relations between a blood pressure difference and a rise time t₁₂, t₂₃, or t₁₃ can be formulated as follows, for example:

k₁₂fₜ(t₁₂)=0.4·(SBP−DBP), (22)

k₂₃f_hd t(t₂₃)=0.4·(SBP−DBP), (23)

k₁₃fₜ(t₁₃)=0.8·(SBP−DBP), (24)

wherein

fₜ(tᵢⱼ)=(t_{i,n}−t_{j,n}−(\hat{t}_{i,m}−\hat{t}_{j,m}))/(\hat{t}_{i,m}−\hat{t}_{j,m}), (25)

and wherein the coefficients k₁₂, k₂₃, and k₁₃ may be determined based on the calibration samples, for example. Based on the Equations (22) to (24), three estimates of relative pulse pressure change PPᵣₑₗ can be calculated as follows, for example:

PPᵣₑₗ₁₂=k₁₂·fₜ(t₁₂)/0.4, (26)

PPᵣₑₗ₂₃=k₂₃·fₜ(t₂₃)/0.4, (27)

PPᵣₑₗ₁₃=k₁₃·fₜ(t₁₃)/0.8. (28)

Based on the Equations (26) to (28), three redundant estimates of pulse pressure PP may be calculated as follows, for example:

PP₁₂=PP_{ref}·(1+PPᵣₑₗ₁₂), (29)

PP₂₃=PP_{ref}·(1+PPᵣₑₗ₂₃), (30)

PP₁₃=PP_{ref}·(1+PPᵣₑₗ₁₃), (31)

wherein

PP_{ref}=SBP_{ref}−DBP_{ref} (32)

The pulse pressures PP₁₂, PP₂₃, and PP₁₃ in Equations (29) to (31) may act as intermediate estimates, and final estimates of the systolic and diastolic blood pressures may then be calculated based on them. For example, a best estimate of the pulse pressure may first be calculated as an average, a median or a weighted average of the intermediate estimates PP₁₂, PP₂₃, and PP₁₃. Preferably the best estimate of the pulse pressure is calculated as a median of the intermediate estimates. Final estimates SBPₑₛₜ and DBPₑₛₜ of the systolic blood pressure and diastolic pressure may then be calculated on the basis of the best estimate PPₑₛₜ of the pulse pressure as follows, for example:

SBPₑₛₜ=(SBP_{ref}+DBP_{ref})/2+PPₑₛₜ/2, (33)

DBPₑₛₜ=(SBP_{ref}+DBP_{ref})/2−PPₑₛₜ/2. (34)

While the first and second exemplary embodiment show the use of three PTTs based on three different reference points, any plurality of PTTs and reference points may be used. The more reference points (such as four, five, or more), the more redundant intermediate estimates can be calculated. Wave analysis of PPG (like decomposition to forward wave and reflection waves) is also feasible from the model of the PPG waveform. Accordingly, in a third exemplary embodiment, the estimation method comprises decomposing the first PPG signal into a wave components, and the timing parameter set comprises at least one (i.e. one or two) of the following timing parameters: a pulse transition time between a forward wave component and a reflection wave component of the first PPG signal, and a pulse transition time between a first reflection wave components and a second reflection wave component of the first PPG signal. The forward wave is caused by heart contraction and reflection wave components originate from junctions of large arteries. The time difference between forward and refection waves can be used to estimate wave velocity and, thus, blood pressure (according to Moens-Korteweg's equation). The third exemplary may also incorporate the features of the first exemplary embodiment.

The monitoring method and estimation method according to the present disclosure are not limited to a single PPG channel. Instead, in a fourth exemplary embodiment, the monitoring method further comprises measuring a second PPG signal from the person (in addition to the first PPG signal) in the measurement phase, and estimation method further comprises receiving the second PPG signal. The pre-processing steps as discussed above may be applied to both PPG channels. The timing parameter set comprises at least one of the following timing parameters: a pulse transition time between ECG signal and the second PPG signal, a pulse transition time between the first PPG signal and the second PPG signal, a rise time calculated on the basis of the second PPG waveform, and a fall time calculated on the basis of the second PPG signal. The PPGs can be measured at two different locations, including: chest, wrist, stomach, arm, and leg.

Preferably, timing parameter set in the fourth exemplary embodiment includes at least the pulse transition time between the first PPG signal and the second PPG signal. As discussed above, the two PPG waveforms represent real pulse wave propagation over a distance and the effects by the peripheral circulation are effectively cancelled out.

The timing parameter set also preferably comprises at least two pulse transition times as defined in the first and fourth exemplary embodiment, since the redundancy facilitates increased accuracy and improves performance. The fourth exemplary may also incorporate the features of the first, second and/or third exemplary embodiment.

In a fifth exemplary embodiment, the estimation method comprises receiving a second PPG signal measured from the person decomposing the second PPG signal into wave components. The timing parameter set comprises at least one of the following timing parameters: a pulse transition time between a forward wave component and a reflection wave component of the second PPG signal, a pulse transition time between a first reflection wave components and a second reflection wave component of the second PPG signal, a pulse transition time between a forward wave component of the first PPG signal and a forward wave component of the second PPG signal, a pulse transition time between a reflection wave component of the first PPG signal and a reflection wave component of the second PPG signal, a pulse transition time between a reflection wave component of the first PPG signal and a forward wave component of the second PPG signal, and a pulse transition time between a forward wave component of the first PPG signal and a reflection wave component of the second PPG signal. The fifth exemplary embodiment may also incorporate the features any one, two or three of the first, second, third and fourth exemplary embodiment.

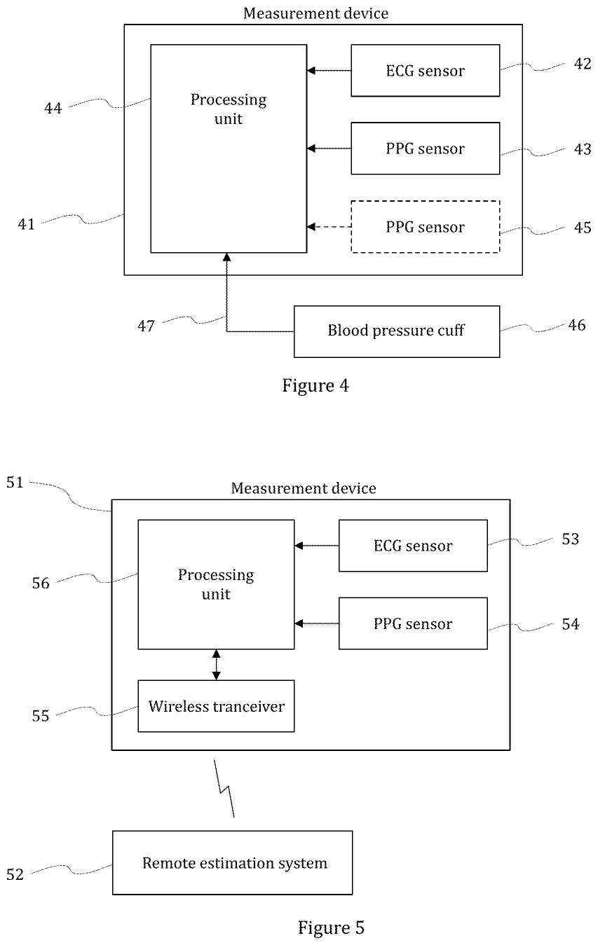

The above-discussed monitor method and estimation method can be implemented in various ways. For example, FIG. 4 shows a simplified block diagram of exemplary embodiment of a measurement arrangement for continuously estimating blood pressure. The arrangement comprises a measurement device 41 that comprises an ECG sensor 42 configured to measure an ECG signal from the person, a first PPG sensor 43 configured to measure a first PPG signal from the person and a processing unit 44. The measurement device 41 may be configured to perform the measurement from the chest of the person. This enables a compact implementation of the device in a single housing.

The processing unit 44 may comprise a processing unit with memory, and is configured to extract values of a timing parameter set based on timings of the ECG signal and the PPG signal, wherein the timing parameter set comprise at least two timing parameters, and calculate intermediate estimates of the blood pressure of the person with linear regression models that are based on different subsets of the timing parameter set. The processing unit 44 is further configured to calculate at least one final estimate on the basis of the intermediate estimates.

The measurement arrangement may further utilize a second PPG measurement. In FIG. 4, the measurement device 41 further comprises an optional second PPG sensor 45 (shown in dashed line) configured to measure a second PPG signal from the person. Further, the measurement arrangement the arrangement may further comprise a blood pressure cuff. In FIG. 4, an external cuff 46 attaches to the measurement device 41 via a dedicated interface. In FIG. 4, this interface 47 is a wired interface. Alternatively, the communication between cuff and the measurement device 41 may be wireless. The cuff may also be integrated to the measurement device.

In FIG. 4, the device 41 acts as stand-alone device. It is able to produce an estimate of the blood pressure based on recorded data on its own.

Alternatively, instead of a stand-alone approach, measured and recorded data may be streamed to a cloud in order to be stored and/or analysed there remotely. The calibration data may also be stored and processed in the cloud.

FIG. 5 shows a simplified block diagram of another exemplary embodiment of a measurement arrangement for continuously estimating blood pressure. In FIG. 5, the measurement arrangement comprises a measurement device 51 and a remote estimation system 52.

The measurement device comprises an ECG sensor 53 configured to measure an ECG signal from the person, a first PPG sensor 54 configured to measure a first PPG signal from the person, a wireless transceiver 55, and a processing unit 56 configured generate ECG and PPG signal data based on the measured ECG signal and first PPG signal and transmit the ECG and PPG signal data to a remote estimation system 52 with the wireless transceiver 55.

The remote estimation system 52 is configured to receive the ECG and PPG signal data from the measurement device 51, extract values of a timing parameter set representing timings of the ECG signal and the PPG signal based on the ECG and PPG signal data, wherein the timing parameter set comprise at least two timing parameters, and calculate intermediate estimates of the blood pressure of the person with linear regression models that are based on different subsets of the timing parameter set. The remote estimation system 52 is further configured to calculate at least one final estimate on the basis of the intermediate estimates. In FIG. 5, the remote estimation system is implemented as a cloud computing system.

Possible uses for the methods and arrangements according to the present disclosure include home monitoring in day and night time and recording of vital signals: ECG, PPG, and possibly others, such as temperature, acceleration etc, to find out a person if is experiencing health conditions that need further diagnosis.

It is obvious to a person skilled in the art that the electrode patch and the detection method/system can be implemented in various ways. The invention and its embodiments are not limited to the examples described above but may vary within the scope of the claims.

1. A method for continuously estimating blood pressure, wherein the method comprises
receiving an electrocardiography (ECG) signal measured from a person;
receiving a first photoplethysmography (PPG) signal measured from the person;
extracting values of a timing parameter set based on timings of the ECG signal and the PPG signal, wherein the timing parameter set comprises at least two timing parameters;
calculating at least two intermediate estimates of the blood pressure of the person with linear regression models that model are based on different timing parameter subsets of the timing parameter set;
calculating at least one final estimate on the basis of the intermediate estimates.

2. A method according to claim 1, wherein the extracting of the values of the timing parameter set comprises
detecting a QRS complex in the ECG signal;
time-aligning the ECG and PPG signals with respect to the detected QRS complex in order to extract ECG and PPG waveform cycles;
averaging the ECG and PPG waveform cycles in order to produce averaged ECG and PPG waveforms, wherein weighting coefficients of the average is adjusted on the basis of a quality of at least one of the ECG and PPG signals; and
determining the values of the timing parameter set on the basis of the averaged ECG and PPG waveforms.

3. A method according to claim 1, wherein each linear regression model has at least one explanatory variable that is weighted by at least one regression model coefficient, wherein
said at least one explanatory variable represents a deviation between a value of a timing parameter in a timing parameter subset and a corresponding calibration timing value determined based on a calibration sample set,
the calibration sample set represents blood pressure characteristics of the person and is based on calibration measurement data of the person, and
said at least one regression model coefficient is based on the calibration sample set.

4. A method according to claim 3, wherein
at least one of the linear regression models is a multiple linear regression model in the form of a weighted sum of a plurality of explanatory variables, wherein each explanatory variable is weighted by a regression model coefficient; and
the timing parameter subset comprises at least two pulse transition times (PTT) between the signal ECG and the first PPG signal, wherein each of said PTTs uses a different threshold level of the PPG waveform as a timing reference point; and wherein the multiple regression model uses said at least two PTTs as explanatory variables.

5. A method according to claim 3, wherein
the timing parameter subset of at least one of the linear regression models comprises a time difference between at least two pulse transition times (PTT) between the signal ECG and the first PPG signal, wherein each of said PTTs uses a different threshold level of the PPG waveform as a timing reference point; and
said regression model uses said time difference as an explanatory variable.

6. A method according to claim 3, wherein the method further comprises
receiving the calibration measurement data, wherein the calibration measurement data comprises the calibration blood pressure values of the person measured with a cuff blood pressure cuff

7. A method according to claim 3, wherein the method comprising
receiving the calibration measurement data before or after the extraction of the values of the timing parameter set.

8. A method according to claim 3, wherein the method comprises
receiving the calibration measurement data during the extraction of the values of the timing parameter set.

9. A method according to claim 8, wherein the method comprise
detecting a change in the values of the timing parameter set,
comparing the change with a set limit, and if the change exceeds the set limit:
generating a trigger signal causing the calibration measurements to be performed in order to update the calibration measurement data,
receiving updated calibration measurement data resulting from the calibration measurements, and
updating the calibration sample set and the regression model coefficients on the basis of the updated calibration measurement data.

10. A method according to claim 1, wherein the timing parameter set comprises at least one of the following timing parameters:
a pulse transition time (PTT) between the signal ECG and the first PPG signal,
a rise time calculated on the basis of the first PPG signal, and
a fall time calculated on the basis of the first PPG signal.

11. A method according to claim 1, wherein the method further comprises receiving a second PPG signal measured from the person, and wherein the timing parameter set comprises at least one of the following timing parameters:
a pulse transition time between ECG signal and the second PPG signal,
a pulse transition time between the first PPG signal and the second PPG signal,
a rise time calculated on the basis of the second PPG waveform, and
a fall time calculated on the basis of the second PPG signal.

12. A method according to claim 1, wherein the method further comprises
decomposing the first PPG signal into a wave components; and
wherein the timing parameter set comprises at least one of the following timing parameters:
a pulse transition time between a forward wave component and a reflection wave component of the first PPG signal, and
a pulse transition time between a first reflection wave components and a second reflection wave component of the first PPG signal.

13. A method according to claim 1, wherein the method further comprises
receiving a second PPG signal measured from the person,
decomposing the second PPG signal into wave components; and
wherein the timing parameter set comprises at least one of the following timing parameters:
a pulse transition time between a forward wave component and a reflection wave component of the second PPG signal,
a pulse transition time between a first reflection wave components and a second reflection wave component of the second PPG signal,
a pulse transition time between a forward wave component of the first PPG signal and a forward wave component of the second PPG signal,
a pulse transition time between a reflection wave component of the first PPG signal and a reflection wave component of the second PPG signal,
a pulse transition time between a reflection wave component of the first PPG signal and a forward wave component of the second PPG signal, and
a pulse transition time between a forward wave component of the first PPG signal and a reflection wave component of the second PPG signal.

14. A monitoring method, wherein the monitoring method comprises
measuring an ECG signal and at least a first PPG signal,
forming an estimate of blood pressure of a person with an estimation method according to claim 1.

15. A measurement arrangement for continuously estimating blood pressure, wherein the arrangement comprises a measurement device that comprises
an ECG sensor configured to measure a ECG signal from the person;
a first PPG sensor configured to measure a first PPG signal from the person; and
a processing unit configured to
extract values of a timing parameter set based on timings of the ECG signal and the PPG signal, wherein the timing parameter set comprises at least two timing parameters;
calculate at least two intermediate estimates of the blood pressure of the person with linear regression models that are based on different timing parameter subsets of the timing parameter set; and
calculating at least one final estimate on the basis of the intermediate estimates.

16. A measurement arrangement according to claim 15, wherein measurement device further comprises
a second PPG sensor configured to measure a second PPG signal from the person.

17. A measurement arrangement according to claim 15, wherein the arrangement further comprises a blood pressure cuff.

18. A measurement arrangement for continuously estimating blood pressure, wherein the arrangement comprises
a measurement device comprising
an ECG sensor configured to measure an ECG signal from the person,
a first PPG sensor configured to measure a first PPG signal from the person,
a wireless transceiver, and
a processing unit configured generate ECG and PPG signal data based on the measured ECG signal and first PPG signal and transmit the ECG and PPG signal data to a remote estimation system with the wireless transceiver; and
a remote estimation system configured to
receive the ECG and PPG signal data from the measurement device,
extract values for a timing parameter set representing timings of the ECG signal and the PPG signal based on the ECG and PPG signal data, wherein the timing parameter set comprise at least two timing parameters; and
calculate at least two estimates of the blood pressure of the person with linear regression models that are based on different timing parameter subsets of the timing parameter set; and
calculating at least one final estimate on the basis of the intermediate estimates.

19. A measurement arrangement according to claim 18, wherein the remote estimation system is implemented as a cloud computing system.
